(19) United States
(12) Patent Application Publication
(10) Pub. No.: US 2004/0242485 A1
(43) Pub. Date: Dec. 2, 2004

(54) Compositions and methods for the amelioration of leptin resistance

(76) Inventors: Takashi Kadowaki, Tokyo (JP); Kazuyuki Tobe, Tokyo (JP); Ryo Suzuki, Tokyo (JP)

Correspondence Address: LAHIVE & COCKFIELD, LLP., 28 STATE STREET, BOSTON, MA, 02109, US

(21) Appl. No.: 10/857,658

(22) Filed: May 28, 2004

Related U.S. Application Data
(60) Provisional application No. 60/474,484, filed on May 30, 2003.

(51) Int. Cl. A61K 38/17; A61K 31/405

(52) U.S. Cl. 514/12; 514/419

(57) ABSTRACT

It is an objective of the present invention to provide a novel method of ameliorating leptin resistance. In order to achieve the above objective, the present invention provides a method of ameliorating leptin resistance in a patient, which comprises a process of administering a β₃ adrenergic receptor agonist to the patient with leptin resistance.

### RELATED APPLICATIONS

This application claims priority to U.S. Provisional Application 60/474,484, entitled “Compositions and Methods for the Amelioration of Leptin Resistance”, filed May 30, 2003. The entire contents of the aforementioned application is hereby incorporated herein by reference.

### BACKGROUND OF THE INVENTION

1. Field of the Invention

The present invention relates to a method of ameliorating leptin resistance.

2. Description of the Related Art

Leptin is secreted from adipose tissues and is a hormone which plays an important role in the regulation of appetite and energy metabolism. Furthermore, it has recently been revealed that leptin is involved in various bodily functions such as reproductive function, hormone secretion, cell growth, immunity, metabolism of sugar, and regulation of blood pressure. The action of leptin is effectuated through leptin receptors, some subtypes of which have been identified. Leptin receptors have been observed in many tissues, and in particular, the leptin receptors in the hypothalamus are thought to be significantly involved in the regulation of appetite.

The leptin concentration in blood changes depending on the physiological conditions. The leptin concentration in blood increases when there is a sufficient energy accumulation (i.e., obese), and the lipolysis in adipose tissues accelerates in conjunction with suppression of appetite. On the other hand, the leptin concentration in blood decreases when there is no sufficient energy accumulation (i.e., non-obese), and the lipolysis in adipose tissues is inhibited in conjunction with an increase in appetite. (Lonnqvist F. et al., Int. J. Med. 245, 643, 1999)

However, in spite of the high levels of leptin concentration in blood in patients with obesity, a phenomenon that the action of leptin is not sufficiently manifested, or the so-called “leptin resistance,” in which sensitivity to leptin is attenuated is observed.

Amelioration of leptine resistance is not only important for the prevention and treatment of obesity, but also for the prevention and treatment of some diseases such as diabetes, hyperlipidemia and arteriosclerosis, which are closely related to obesity. For example, a method of ameliorating leptin resistance by administration of diacylglycerol is known in the field. (Japanese Patent Pub. No. 2002-3376)

On the other hand, of the three types of adrenergic β₃-receptors (β₁,β₂,β₃), the β₃-receptors are known to exist on brown adipose cells and to function in calorie production by lipolysis of white adipose tissues adhering to the subcutis and visceral organs via Uncoupling Protein 1 (UCP1) by means of the action of the β₃-adrenergic receptor agonist binding to the receptor. Moreover, it is suggested that β₃-adrenergic receptors are linked to diabetes and to non-insulin dependant diabetes (type-II diabetes). For example, it has been reported that β₃-adrenergic receptor agonists exert anti-diabetes and anti-obesity actions in rodents of the type II diabetes model. (Carroll M J et al., Diabetes 34, 1198-1204, 1985; Cawthorne M A et al., Int. J. Obes. 8 (Suppl. 1), 93-102, 1984; Largis E E et al., Drug Dev. Res. 32, 69-76, 1994; Yoshida T. et al., Life Sci. 54, 491-498, 1994) However, the mechanisms of anti-diabetes and anti-obesity actions by β₃-adrenergic receptor agonists are not clear, and moreover, the relationship between the β₃-adrenergic receptors agonists and leptin resistance is not clear either.

### SUMMARY OF THE INVENTION

First, it is an objective of the present invention to provide a novel method of ameliorating leptin resistance.

Second, it is an objective of the present invention to provide a pharmaceutical composition, which can ameliorate leptin resistance and/or enhance the action of leptin in conjunction with said amelioration of leptin resistance.

Third, it is an objective of the present invention to provide a pharmaceutical composition, which reduces the weight-gaining effect of a pharmaceutical agent by means of ameliorating leptin resistance in relation to said pharmaceutical agent having a side effect of weight gain in addition to its intended prevention and/or treatment effects.

In order to achieve the above objectives, the present invention provides a method of ameliorating leptin resistance comprising a process of administering an effective dose of a β₃-adrenergic receptor agonist to a patient with leptin resistance.

Moreover, in order to achieve the above objectives, the present invention provides a pharmaceutical composition comprising a β₃-adrenergic receptor agonist and/or leptin, in a therapeutically effective amount capable of ameliorating leptin resistance.

Furthermore, in order to achieve the above objectives, the present invention provides a pharmaceutical composition comprising a β₃-adrenergic receptor agonist and an agent having a side effect of weight gain in addition to its intended prevention and/or treatment effects.

### BRIEF DESCRIPTION OF THE DRAWINGS

FIG. 1 is a graph showing the changes in body weight (%) in cases of intraperitoneal administration of leptin to wild-type and IR S-2 knockout mice.

FIG. 2 is a graph showing the changes in body weight (%) in cases of cranial ventricular administration of leptin to wild-type and IR S-2 knockout mice.

FIG. 3 is a set of graphs showing the amounts of adipose tissues (epididymal adipose cells/body weight (mg/g)), and the amounts of leptin concentrations in blood (ng/ml).

FIG. 4 is a set of graphs showing the changes in the amounts of food intake (%) and in body weight (%) in cases of intraperitoneal administration of leptin after the administration of β₃-adrenergic receptor agonists to IR S-2 knockout mice.

FIG. 5 is an illustration of a method of producing an IR S-2 knockout mouse.

### DESCRIPTION OF THE PREFERRED EMBODIMENTS

In the present invention, “leptin resistance” means a physiological phenomenon in which sensitivity to leptin is reduced. Note that, here, the term “patient” mainly corresponds to “human,” and may correspond to “primates” including human, mammals, etc. Therefore, in a patient with leptin resistance, even if a sufficient amount of leptin is present in blood, the physiological effects of leptin are not sufficiently manifested. Examples of physiological effects of leptin include eating suppression, an increase in energy expenditure, amelioration of obesity, amelioration of insulin-resistance, etc. In a patient with leptin resistance, the above physiological effects of leptin are reduced. Examples include patients with obesity, over-eaters of high-fat food, type-II diabetics, etc. For example, large portion of patients with obesity (90˜95%) exhibits symptoms of hyperleptinemia, in which the physiological effect of leptin is not sufficiently manifested despite the leptin concentration in blood is at a high level. The possible causes for leptin resistance may include a disorder of transportation of leptin through the blood-brain barrier from blood to the brain, a decrease in sensitivity of leptin receptors, a malfunction of leptin receptors, an abnormal signal transmission in relation to leptin receptors (e.g. phosphorylation of STAT3, and a decrease in PI3 kinase activation), an abnormal melanocortin 4-receptor gene and an abnormal POMC gene which encodes the ligand thereof, an over-expression of NPY which is a feeding-promoting peptide, etc. It is thought that the β₃ adrenergic receptor agonist ameliorates leptin resistance by ameliorating one or two or more of the above possible causes for leptin resistance. By ameliorating leptin resistance in a patient with leptin resistance, the original physiological effects of leptin are manifested so that, for example, weight reduction, etc. occur with suppression of eating, an increase in energy expenditure, etc. Therefore, by manifesting the above original physiological effects of leptin, it becomes possible to prevent or treat obesity, and to prevent or treat diabetes, hyperlipidemia and arteriosclerosis, which are closely related to obesity.

In the present invention, the type of β₃ adrenergic receptor agonists administered to a patient with leptin resistance is not particularly limited, and may be of any substances, which directly or indirectly have actions as a β₃ adrenergic receptor agonist. Moreover, the β₃ adrenergic receptor agonist, which is administered to the patient with leptin resistance, may be used by combining one or more of β₃ adrenergic receptor agonists as long as it has the action of the β₃ adrenergic receptor agonist.

Note that the above described leptin may be a leptin fragment, or a product of enzymolysis, which manifests the action of leptin. The above described leptin only has to include a necessary structure for leptin activity. Moreover, the above described leptin may be a synthesized peptide which shows leptin activity.

The β₃ adrenergic receptor agonist will be described hereinafter, but first the terms used herein will be defined as below.

A “lower alkyl group” or “lower alkyl” corresponds to an alkyl group, which is a straight- or branched-chain of one to four carbons. Examples thereof include a methyl group, an ethyl group, a propyl group, an isopropyl group, a buthyl group, an isobuthyl group, a sec-buthyl group, a tert-buthyl group, etc.

A “lower alkoxy group” or “lower alkoxy” corresponds to an alkoxy group, which is a straight- or branched-chain of one to four carbons. Examples thereof include a methoxy group, an ethoxy group, a propoxy group, an isopropoxy group, a butoxy group, an isobutoxy group, a sec-butoxy group, a tert-butoxy group, etc.

Examples of a “lower alkyl group which is substituted with a hydroxyl group” include a hydroxymethyl group, a 2-hydroxymethyl group, etc.

Examples of a “lower alkylsulfonylamino group” include a methylsulfonylamino group, an ethylsulfonylamino group, etc.

Examples of a “mono- or di-lower alkylaminosulfonyl group” include monomethylaminosulfonyl group, a dimethylaminosulfonyl group, a monoethylaminosulfonyl group, a diethylaminosulfonyl group, etc.

Examples of a “lower alkoxycarbonyl group” include a methoxycarbonyl group, an ethoxycarbonyl group, a propoxycarbonyl group, a buthoxycarbonyl group, etc.

Examples of a “mono- or di-lower alkylaminocarbonyl group” include a methylaminocarbonyl group, a dimethylaminocarbonyl group, a methylethylaminocarbonyl group, etc.

Examples of a “lower-alkoxycarbonyl-lower-alkoxy group” include a methoxycarbonylmethoxy group, a 1-methoxycarbonylethoxy group, a 2-methoxycarbonylethoxy group, a 1-methoxycarbonylpropoxy group, a 3-methoxycarbonylpropoxy group, etc.

Examples of a “carboxy-lower-alkoxy group” include a carboxymethoxy group, a 1-carboxyethoxy group, a 2-carboxyethoxy group, a 1-carboxypropoxy group, a 3-carboxypropoxy group, etc.

Examples of a “phenyl-lower-alkoxy group” include a benzyloxy group, a phenethyloxy group, etc.

A “halogen atom” corresponds to a fluorine, chlorine, bromine or iodine atom. Examples of a preferable halogen atom include a fluorine, chlorine, or bromine atom.

A “cyclic amino” corresponds to a cyclic amine of a 5-membered or 6-membered ring, which includes at least one nitrogen atom and may include oxygen atoms. Examples thereof include pyrrolidine, piperidine, morpholine, etc.

A “lower alkanoyl group” corresponds to a straight- or branched-chain of one to five carbons. The examples thereof include a formyl group, an acetyl group, a propionyl group, a buthylyl group, an isobuthylyl group, a valeryl group, a pivaloyl group, etc.

“Salt” corresponds to acid addition salt which is physiologically permissible, alkali metal salt, alkaline earth metal salt, or salt with organic base. Examples of acid addition salt include inorganic acid salt such as hydrochloride, hydrobromide, hydroiodide, hydrosulfate, and phosphate; and organic acid salt such as oxalate, maleate, fumarate, malonate, lactate, malate, citrate, tartrate, benzoate, metane sulfonic acid salt, p-toluenesulfonic acid salt, and gluconic acid salt. Examples of alkali metal salt include inorganic alkaline salt such as sodium salt, and potassium salt. Examples of alkaline earth metal salt include calcium salt and magnesium salt. Examples of salt with organic base include salt of ammonia, methylamine, triethylamine, tributhylamine, diisopropylethylamine, N-methylmorpholine, and dicyclohexylamine.

An example of a β₃ adrenergic receptor agonist includes a compound or the salt thereof, which is represented by the following formula (I). Note that the method of producing the compound, which is represented by the following formula (I), and the activity of the β₃ adrenergic receptor agonist are disclosed in WO96/16938, which is incorporated herein by reference.

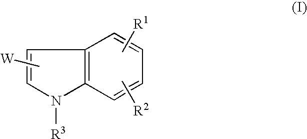

In the formula (I), R¹ represents the following groups, in which a hydroxyl group can be substituted, respectively: a lower alkyl group, a phenylsulfonylamino group, a lower alkylsulfonylamino group, a mono- or di-lower alkylaminosulfonyl group, a group which is selected from following formulas (a) to (d), or a methylenedioxy group, which may be substituted by a carboxyl group or a lower alkoxycarbonyl group in conjunction with R²:

(a) a group which is represented by “—X—R^{a},” (Here, X represents O, S, or NH, and R^{a} represents a hydrogen atom or a lower alkyl group. However, if X═S, R^{a} is a lower alkyl group);

(b) a group which is represented by the following formula:

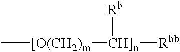

(Here, R^{b} represents a hydrogen atom, a lower alkyl group, a lower alkoxycarbonyl group, or a carboxyl group. R^{bb} represents a lower alkoxycarbonyl group or a carboxyl group. m represents an integral number of 0 to 3. n represents an integral number of 0 or 1);

(c) a group which is represented by “—O(CH₂)ₚ—R^{c},” (Here, R^{c} represents a lower alkanoyl group, a hydroxyl group, a cyano group, a phenyl group, a mono- or di-lower alkylaminocarbonyl group, or a group which is represented by the following formula:

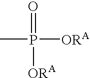

Wherein, p represents an integral number of 1 to 4. Here, R^{A} is a hydrogen atom or a lower alkyl group); and

(d) a group selected from the groups which are represented by “—Y—(CH₂)_{q}—R^{d},” (Here, Y represents NH or S. R^{d} represents a carboxyl group or a lower alkoxycarbonyl group. q represents an integral number of 1 to 4.)

R² represents a lower alkyl group, a hydroxyl group, or a lower alkoxy group, in which a hydrogen atom, a halogen atom, or a hydroxyl group can be substituted; the same group as above (b) or (c); or a methylenedioxy group which may be substituted with a carboxyl group or a lower alkoxycarbonyl group in conjunction with R¹.

R³ represents a hydrogen atom or a lower alkyl group.

W represents a group of the following formula (I′), which is bonded to the second or the third position of the indole ring of the above formula (I):

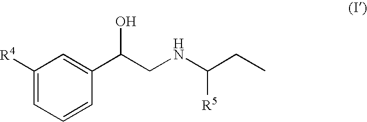

Here, R⁴ is a halogen atom or a trifluoromethyl group, and R⁵ is a lower alkyl group.

A compound represented by the above formula (I) has two asymmetric carbons. Therefore, in the formula (I), the carbon atom to which the hydroxyl group is bonded, and the carbon atom to which R⁵ is bonded are asymmetric carbons. Therefore, in a compound represented by the above formula (I), four types of stereoisomers can exist. Moreover, a compound represented by the above formula (I) may be one type of stereoisomer, or may be a mixture of more than two types of stereoisomers. Moreover, a compound represented by the above formula (I) may be any one of optically-active substances, racemic bodies, or the mixtures thereof.

A preferable compound of the compounds which are represented by the above formula (I) is a compound or the salt thereof, which is represented by the following formula (I-a), in which substituent W is bonded to the third position of the indole ring of the formula (I):

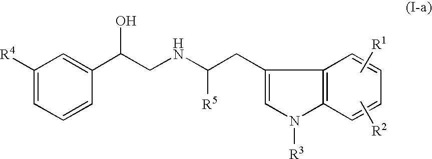

In the formula (I-a), R¹, R² ,R³,R⁴ , and R⁵ are the same as the above.

In a preferable compound or the salt thereof, which is represented by the above formula (I-a), R¹ is bonded to the fifth, the sixth, or the seventh position of the indole ring, and R² is a hydrogen atom; or alternatively R² is a group other than a hydrogen atom, and any one of R¹ or R² is bonded to the sixth position of the indole ring and the other is bonded to the seventh position thereof.

Moreover, of the compounds represented by the above formula (I-a), a compound or the salt thereof is preferable, in which R¹ represents a lower alkyl group, a phenylsulfonylamino group, a lower alkylsulfonylamino group, a di-lower alkylaminosulfonyl group, which is respectively substituted with a hydroxyl group, or a group which is selected from (a), (b-1), (c-1), and (d); and R² represents a hydrogen atom, a halogen atom, a lower alkyl group which may be substituted with a hydroxyl group, a hydroxyl group, a lower alkoxy group, or the same group as (b-1) or (c-1):

(a) a group which is represented by “—X—R^{a},” (Here, X and R^{a} are the same as the above.);

(b-1) a group which is represented by the following formula:

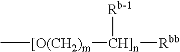

(Here, R^{b-1} represents a hydrogen atom or a lower alkyl group, R^{bb}, m, and n are the same as the above.);

(c-1) a group which is represented by “—O(CH₂)p —R^{c-1},” (Here, R^{c-1} represents a lower alkanoyl group, a phenyl group, or a mono-lower alkylaminocarbonyl group. p is the same as the above.); and

(d) a group which is represented by “—Y— (CH₂)q —R^{d},” (Here, Y, R^{d}, and q are the same as the above.)

In the above compounds or salts thereof, a compound or the salt thereof is preferable, in which R¹ represents a lower alkoxy group, a lower alkyl group, a lower alkoxycarbonyl group, a lower alkoxycarbonyl-lower-alkoxy group, a carboxy-lower-alkoxy group, a phenyl-lower-alkoxy group, or a di-lower-alkylaminosulfonyl group; R² represents a hydrogen atom, a halogen atom, a lower alkoxy group, a lower alkyl group, a lower alkoxycarbonyl group, a lower alkoxycarbonyl-lower-alkoxy group, a carboxy-lower-alkoxy group, a phenyl-lower-alkoxy group; R³ represents a hydrogen atom; R⁴ represents a halogen atom; and R⁵ represents a methyl group. Moreover, a compound or the salt thereof is particularly preferable, in which R¹ represents a methoxy group, an ethoxy group, a propoxy group, an isopropoxy group, a methyl group, a methoxycarbonyl group, a methoxycarbonylmethoxy group, a carboxymethoxy group, a benzyloxy group, or a dimethylaminosulfonyl group; R² represents a hydrogen atom or a methoxy group; R³ represents a hydrogen atom; R⁴ represents a chlorine atom; and R⁵ represents a methyl group.

The most preferable compound is a compound or the salt thereof, in which, in the above formula (I-a), R¹ represents a methoxy group, an ethoxy group, a methoxycarbonyl group, a methoxycarbonylmethoxy group, or carboxymethoxy group, which respectively is bonded to the sixth or the seventh position of the indole ring; R² and R³ represents respectively a hydrogen atom; R⁴ represents a chlorine atom; and R⁵ represents a methyl group.

Examples of the compounds represented by the above formula (I) include

3-(2-(3-chlorophenyl-2-hydroxyethylamino)propyl)indole-7-yloxyacetic acid (hereinafter referred to as “AJ-9677”),

3-[2-(3-(2-chlorophenoxy)-2-hydroxypropylamino)propyl]indole-7-yloxyacetic acid,

3-[2-(3-(4-hydroxyphenoxy)-2-hydroxypropylamino)propyl]indole-7-yloxyacetic acid methylester,

3-(2-(3-phenoxy-2-hydroxypropylamino)propyl)-7-benzyloxyindole, 2-(3-(7-methoxyindole-3-yl)-2 propylamino)-1-(3-chlorophenyl)ethanol,

2-(3-(7-ethoxyindole-3-yl)-2-propylamino)-1-(3-chlorophenyl)ethanol,

2-(3-(7-methoxycarbonyl-methoxyindole-3-yl)-2-propylamino)-1-(3-chlorophenyl)ethanol,

2-(3-(7-carboxymethoxyindole-3-yl)-2-propylamino)-1-(3-chlorophenyl)ethanol, etc. Of the above compounds, examples of preferable compounds include

3-(2-(3-chlorophenyl-2-hydroxyethylamino)propyl)indole-7-yloxyacetic acid,

3-[2-(3-(2-chlorophenoxy)-2-hydroxypropylamino)propyl]indole-7-yloxyacetic acid,

3-[2-(3-(4-hydroxyphenoxy)-2-hydroxypropylamino)propyl]indole-7-yloxyacetic acid methylester, and

3-(2-(3-phenoxy-2-hydroxypropylamino)propyl)-7-benzyloxyindole.

Another example of a β₃ adrenergic receptor agonist includes a compound or the salt thereof, which is represented by the following formula (II). Note that the method of producing a compound which is represented by the following formula (II), and the activity of the β₃ adrenergic receptor agonist are disclosed in Japanese Patent Pub. No. 2001-39948, which is incorporated herein by reference.

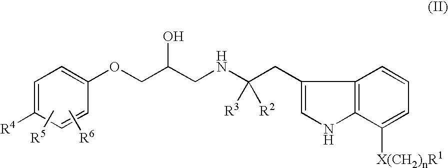

In the formula (II), X represents O, NH, or S. R¹ represents a hydoroxy group, a lower alkoxycarbonyl group, a mono- or di-lower alkylaminocarbonyl group, a carbamoyl group, a cyclic aminocarbonyl group, a carboxyl group, a sulfamoyl group, a mono- or di-lower alkylaminosulfonyl group, a cyclic aminosulfonyl group, a sulfonic acid group, a sulfonic acid ester group, a phosphoric acid group, a phosphoric acid ester group, or a substituted or unsubstituted phenyl group (this substituent is of one or two types of substituents, which is selected from a halogen atom, a trifluoromethyl group, a lower alkyl group, a lower alkoxy group, a lower alkoxycarbonyl group, a carboxyl group, a nitro group, a cyano group, an amino group, and a mono- or di-lower alkylamino group). R² and R³ represent respectively a hydrogen atom or a lower alkyl group, which may be identical or different. R⁴ represents a hydrogen atom or OR⁷. Here, R⁷ represents a hydrogen atom, a lower alkyl group, a substituted or unsubstituted benzyl group (this substituent is of one or two types of substituents, which is selected from a halogen atom, a trifluoromethyl group, a lower alkyl group, a lower alkoxy group, a lower alkoxycarbonyl group, a carboxyl group, a nitro group, a cyano group, an amino group, and a mono- or di-lower alkylamino group), or a lower alkanoyl group. R⁵ represents a hydrogen atom, a halogen atom, a lower alkyl group, a lower alkoxy group, a hydroxy group, a nitro group, a cyano group, an amino group, a mono- or di-lower alkylamino group, a lower alkylsulfonylmino group, or a lower alkylsulfonyl group. R⁶ represents a hydrogen atom, a halogen atom, a lower alkyl group, a lower alkoxy group, a hydroxy group, a nitro group, a cyano group, an amino group, a mono- or di-lower alkylamino group, a lower alkylsulfonylamino group, or a lower alkylsulfonyl group. n represents 1, 2, 3, 4 or 5.

A compound which is represented by the above formula (II) has one or two asymmetric carbons. Therefore, the carbon atom to which the hydroxy group is bonded is an asymmetric carbon. Moreover, in cases where R² and R³ are different groups from each other, the carbon to which these groups are bonded is an asymmetric carbon. Therefore, in cases where, in the above formula (II), R² and R³ are the same atoms or the same groups, two types of stereoisomers can exist, and in cases where R² and R³ are different groups, four types of stereoisomers can exist. A compound which is represented by the above formula (II) may be any one type of stereoisomer, or may be a mixture of more than two types of stereoisomers.

In the compounds which are represented by the above formula (II), in cases where R² and R³ are different groups, four types of stereoisomers can exist. However, it is preferable to have a compound, in which the stereo-configuration of the carbon atom to which the hydroxyl group is bonded is S-configuration, and the stereo-configuration of the carbon atom to which R² and R³ are bonded is R-configuration.

In the compounds which are represented by the above formula (II), a compound, in which X is O, is preferable. A compound, in which X is O, is preferable, wherein R¹ corresponds to a lower alkoxycarbonyl group, a mono- or di-lower alkylaminocarbonyl group, a cyclic aminocarbonyl group, a carboxyl group, a sulfamoyl group, a mono- or di-lower alkylaminosulfonyl group, a cyclic aminosulfonyl group, a sulfonic acid group, or a substituted or unsubstituted phenyl group (this substituent is one or two types of substituents, which is selected from a halogen atom, a trifluoromethyl group, a lower alkyl group, a lower alkoxy group, a lower alkoxycarbonyl group, a carboxyl group, a nitro group, a cyano group, an amino group, and a mono- or di-lower alkylamino group); R² and R³ corresponds respectively to a hydrogen atom, a methyl group, or an ethyl group, which may be identical or different; R⁴ corresponds to a hydrogen atom or OR⁷; R⁷ corresponds to a hydrogen atom, a methyl group, a benzyl group or a lower alkanoyl group; and n corresponds to 1, 2 or 3.

Examples of the compounds which are represented by the above formula (II) include

3-(2-(3-phenoxy-2-hydroxypropylamino)propyl)indole-7-yloxy acetic acid, 3-(2-(3-phenoxy-2-hydroxypropylamino)propyl)indole-7-benzyloxyindole, 3-(2-(3-phenoxy-2-hydroxypropylamino)propyl)indole-7-yloxy acetic acid methylester, 3-[2-(3-(4-hydroxyphenoxy)-2-hydroxypropylamino)propyl]-7-benzyloxyindole, 3-[2-(3-(4-hydroxyphenoxy)-2-hydroxypropylamino)propyl]-7-((4-methoxycarbonylphenyl)methoxy) indole, 3-[2-(3-(4-hydroxyphenoxy)-2-hydroxypropylamino)propyl]-7-((4-carboxyphenyl)methoxy)indole, 3-[2-(3-(4-hydroxyphenoxy)-2-hydroxypropylamino)propyl]indole-7-yloxy acetic acid, 3-[2-(3-(4-hydroxyphenoxy)-2-hydroxypropylamino)propyl]indole-7-yloxyacetic acid-N,N-diethylamido, 3-[2-(3-(4-hydroxyphenoxy)-2-hydroxypropylamino)propyl]indole-7-yloxya acetic acid methylester, 3-[2-(3-(2-chlorophenoxy)-2-hydroxypropylamino)propyl]indole-7-yloxy acetic acid, etc.

The other examples of the β₃ adrenergic receptor agonist include compounds and the salts thereof, which are represented by the following formula (III). Note that the method of producing a compound which is represented by the following formula (III), and the activity of the β₃ adrenergic receptor agonist are disclosed in J. Med. Chem., vol. 35, p3081-3084 (1992), which is incorporated herein by reference.

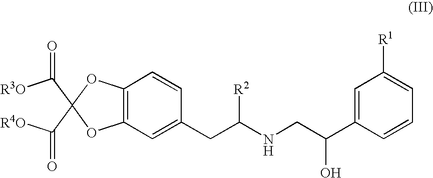

In the formula (III), R¹ represents a halogen atom; R² represents a hydrogen atom or a lower alkyl group; and R³ and R⁴ represent respectively a hydrogen atom or a lower alkyl group, which may be identical or different.

In particular, Sigma Company, Ltd. has introduced in the marketplace under the name of CL-316243 a compound in the above formula (III), in which R¹ is a chlorine atom; R² is a methyl group; and R³ and R⁴ are sodium atoms.

A compound which is represented by the above formula (III) has one or two asymmetric carbons. Therefore, the carbon atom to which the hydroxyl group is bonded is an asymmetric carbon, and in cases where R² is a lower alkyl group, the carbon to which R² is bonded is an asymmetric carbon. Hence, in cases where, in the above formula (III), R² is a hydrogen atom, the carbon atom to which the hydroxy group is bonded is an asymmetric carbon, so that two types of stereoisomers exist with respect to the entire molecule. Moreover, in cases where R² is a lower alkyl group, four types of stereoisomers can exist, the compound which is represented by the above formula (III) may be any one of stereoisomers, or may be a mixture of more than two types of stereoisomers.

The other examples of the β₃ adrenergic receptor agonist include compounds and the salts thereof, which are represented by the following formula (IV). Note that the method of producing a compound, which is represented by the following formula (IV), and the activity of the β₃ adrenergic receptor agonist are disclosed in Japanese Patent Pub. No. 2000-239225, which is incorporated herein by reference.

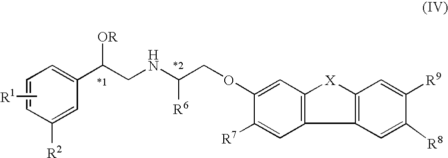

In the above formula (IV), R represents a hydrogen atom or a methyl group; R¹ represents a hydrogen atom, a halogen atom, a hydroxyl group, a benzyloxy group, an amino group, or a hydroxymethyl group; R² represents a hydrogen atom, a hydroxymethyl group, NHR³, SO₂NR⁴R^{4′}, or a nitro group; R³ represents a hydrogen atom, a methyl group, SO₂R⁵, a formyl group, or CONHR⁶; R⁵ represents a lower alkyl group, a benzyl group, or NR⁴R^{4′}; R⁴ and R^{4′} respectively represents a hydrogen atom, a lower alkyl group, or a benzyl group, which may be identical or different; R^{6′} represents a hydrogen atom or a lower alkyl group; R⁶ represents a hydrogen atom or a lower alkyl group; and X represents a secondary nitrogen atom, an oxygen atom, a sulfur atom, or a methylene group. In cases where X is a secondary nitrogen atom, an oxygen atom, or a sulfur atom, R⁹ is a hydrogen atom, and one of R⁷ and R⁸ is a hydrogen atom and the other is a hydrogen atom, an amino group, an acetylamino group or a hydroxyl group. In cases where X is a methylene group, both R⁷ and R⁸ are hydrogen atoms, and R⁹ is a hydrogen atom, an amino group, an acetylamino group or a hydroxyl group. *1 represents an asymmetric carbon atom. In cases where R⁶ is a lower alkyl group, *2 represents an asymmetric carbon atom.

In the above formula (IV), *1 is an asymmetric carbon atom, and in cases where R⁶ is a lower alkyl group, *2 is also an asymmetric carbon atom. In that case, the compound, which is represented by the above formula (IV), can exist as four types of isomers, represented in the order of *1 and *2, i.e., (R, R), (R, S), (S, S), and (S, R). Moreover, in cases where R⁶ is a hydrogen atom, two types of isomers can exist. The compound, which is represented by the above formula (IV), may be a pure optical isomer, or may be a mixture of more than two types of isomers. In terms of pharmacological activities, a preferable configuration of the asymmetric carbon (*1) of the ethanolamino chain is an absolute configuration (R). In particular, for N-[5-[2-[2-(9H-carbazole-2-yloxy)ethylamino]-1-hydroxyethyl]-2-hydroxy phenyl]methansulfonamide, R-hydroxy is preferable at the asymmetric carbon (*1) therein.

In the compounds represented by the above formula (IV), in cases where R² is a hydroxymethyl group, NHR³, SO₂NR⁴R^{4′}, or a nitro group, a preferable substitution position of R¹ is the fourth position or the fifth position, or preferably the fourth position. In cases where R² is a hydrogen atom, a preferable substitution position of R¹ is the second position.

In the compounds represented by the above formula (IV), preferable groups are formed by combining various substituent groups. Below is a list of preferable groups, and unless stated otherwise, R⁶, X, R⁷, R⁸, R⁹, *1 and *2 are the same as the above.

In one example, R represents a hydrogen atom. R¹ represents a hydrogen atom, a halogen atom, a hydroxyl group, a benzyloxy group, an amino group, or a hydroxymethyl group. R² represents a hydrogen atom, a hydroxymethyl group, NHR³, SO₂NR⁴R^{4′}, or a nitro group; R³ represents a hydrogen atom, a methyl group, SO₂R⁵, a formyl group, or CONHR^{6′}; R⁵ represents a lower alkyl group, a benzyl group, or NR⁴R^{4′}; R⁴ and R^{4′} respectively represent a hydrogen atom, a lower alkyl group, or a benzyl group, which may be identical or different; and R^{6′} is the same as the above.

In another example, R represents a hydrogen atom. R¹ represents a hydrogen atom, a fluorine atom, a chlorine atom, a hydroxyl group, or a benzyloxy group. R² represents a hydrogen atom, a hydroxymethyl group, NHR³, SO₂NR⁴R^{4′}, or a nitro group′ R³ represents a hydrogen atom, a methyl group, SO₂R⁵, a formyl group, or CONHR^{6′}; and R⁵ represents a lower alkyl group, a benzyl group, or a dimethylamino group. Any one of R⁴ and R^{4′} represents a hydrogen atom, and the other represents a hydrogen atom, a lower alkyl group, or a benzyl group. R^{6′} is the same as the above.

In another example, R represents a hydrogen atom. R¹ represents a hydrogen atom, a halogen atom, a hydroxyl group, or a benzyloxy group. R² represents a hydroxymethyl group, NHR³, SO₂NR⁴R^{4′}, or a nitro group. R³ represents a hydrogen atom, a methyl group, SO₂R⁵, a formyl group, or CONHR^{6′}. R⁵ represents a lower alkyl group, a benzyl group, or NR⁴R^{4′}. R⁴ and R^{4′} respectively represents a hydrogen atom, a lower alkyl group, or a benzyl group, which may identical or different. R^{6′} is the same as the above.

In another example, R represents a hydrogen atom. R¹ represents a hydrogen atom, a fluorine atom, a chlorine atom, a hydroxyl group, or a benzyloxy group. R² represents a hydroxymethyl group, NHR³, SO₂NR⁴R^{4′}, or a nitro group. R³ represents a hydrogen atom, a methyl group, SO₂R⁵, a formyl group, or CONHR^{6′}. Any one of R⁴ and R^{4′} represents a hydrogen atom, and the other represents a hydrogen atom, a lower alkyl group or a benzyl group. R⁵ represents a lower alkyl group, a benzyl group, or a dimethylamino group. R^{6′} is the same as the above.

In another example, R¹ and R^{1′} respectively represents a hydrogen atom. R² represents a hydroxymethyl group, NHR³, or SO₂NR⁴R^{4′}. R³ represents a hydrogen atom, a methyl group, SO₂R⁵, a formyl group, or CONHR^{6′}. R⁵ represents a lower alkyl group, a benzyl group, or NR⁴R⁴. R⁴ and R^{4′} respectively represents a hydrogen atom, a lower alkyl group, or a benzyl group, which may be identical or different. R^{6′} is the same as the above.

In another example, R¹ and R^{1′} represents a hydrogen atom. R² represents a hydroxymethyl group, NHR³, or SO₂NR⁴R^{4′}. R³ represents a hydrogen atom, a methyl group, SO₂R⁵, a formyl group, or CONHR^{6′}. Any one of R⁴ and R^{4′} represents a hydrogen atom, and the other represents a hydrogen atom, a lower alkyl group or a benzyl group. R⁵ represents a lower alkyl group, a benzyl group, or a dimethylamino group. R^{6′} is the same as the above.

In another example, R represents a hydrogen atom. R¹ represents a halogen atom, or a hydroxyl group. R² represents NHSO₂R⁵, or SO₂NR⁴R^{4′}. R⁵ represents a lower alkyl group, a benzyl group, or NR⁴R^{4′}. R⁴ and R^{4′} respectively represents a hydrogen atom, a lower alkyl group, or a benzyl group, which may be identical or different.

In another example, R represents a hydrogen atom. R¹ represents a fluorine atom, a chlorine atom, or a hydroxyl group. R² represents NHSO₂R⁵, or SO₂NR⁴R^{4′}. Any one of R⁴ and R^{4′} represents a hydrogen atom, and the other represents a hydrogen atom, a lower alkyl group or a benzyl group. R⁵ represents a lower alkyl group, a benzyl group, or a dimethylamino group.

In another example, both of R and R² represent a hydrogen atom, and R¹ represents a hydrogen atom, a halogen atom, or a hydroxyl group.

In another example, both of R and R² express a hydrogen atom, and R¹ represents a hydrogen atom, a fluorine atom, a chlorine atom, or a hydroxyl group.

In another example, R represents a hydrogen atom. R¹ represents a hydrogen atom, a halogen atom, a hydroxyl group, an amino group, or a hydroxymethyl group. R² represents NHR³, or SO₂NR⁴R^{4′}. R³ represents SO₂R⁵. R⁵ represents a lower alkyl group, a benzyl group, or NR⁴R^{4′}. R⁴ and R^{4′} respectively represents a hydrogen atom, a lower alkyl group, or a benzyl group, which may be identical or different.

In another example, R represents a methyl group. R¹ represents a hydrogen atom, a halogen atom, a hydroxyl group, a benzyloxy group, an amino group, or a hydroxymethyl group. R² represents a hydrogen atom, a hydroxymethyl group, NHR³, SO₂NR⁴R^{4′}, or a nitro group. R³ represents a hydrogen atom, a methyl group, SO₂R⁵, a formyl group, or CONHR^{6′}. R⁵ represents a lower alkyl group, a benzyl group, or NR⁴R^{4′}. R⁴ and R^{4′} respectively represents a hydrogen atom, a lower alkyl group, or a benzyl group, which may be identical or different.

In another example, R represents a methyl group. R¹ represents a hydrogen atom, a fluorine atom, a chlorine atom, a hydroxyl group, or a benzyloxy group. R² represents a hydrogen atom, a hydroxymethyl group, NHR³, SO₂NR⁴R^{4′}, or a nitro group. R³ represents a hydrogen atom, a methyl group, SO₂R⁵, a formyl group, or CONHR^{6′}. R⁵ represents a lower alkyl group, a benzyl group, or NR⁴R^{4′}. Any one of R⁴ and R^{4′} represents a hydrogen atom, and the other represents a hydrogen atom, a lower alkyl group or a benzyl group. R^{6′} is the same as the above.

In another example, R represents a methyl group. R¹ represents a hydrogen atom, a halogen atom, a hydroxyl group, or a benzyloxy group. R² represents a hydroxymethyl group, NHR³, SO₂NR⁴R^{4′}, or a nitro group. R³ represents a hydrogen atom, a methyl group, SO₂R⁵, a formyl group, or CONHR^{6′}. R⁵ represents a lower alkyl group, a benzyl group, or NR⁴R^{4′}. R⁴ and R^{4′} respectively represent a hydrogen atom, a lower alkyl group, or a benzyl group, which may be identical or different. R^{6′} is the same as the above.

In another example, R represents a methyl group. R¹ represents a hydrogen atom, a fluorine atom, a chlorine atom, a hydroxyl group, or a benzyloxy group. R² represents a hydroxymethyl group, NHR³, SO₂NR⁴R^{4′}, or a nitro group. R³ represents a hydrogen atom, a methyl group, SO₂R⁵, a formyl group, or CONHR^{6′}. Any one of R⁴ and R^{4′} represents a hydrogen atom, and the other represents a hydrogen atom, a lower alkyl group or a benzyl group. R⁵ represents a lower alkyl group, a benzyl group, or a dimethylamino group. R^{6′} is the same as the above.

In another example, R represents a methyl group. R¹ represents a hydrogen atom. R² represents a hydroxymethyl group, NHR³, or SO₂NR⁴R ^{4′}. R³ represents a hydrogen atom, a methyl group, SO₂R⁵, a formyl group, or CONHR^{6′}. R⁵ represents a lower alkyl group, a benzyl group, or NR⁴R^{4′}. R⁴ and R^{4′} respectively represent a hydrogen atom, a lower alkyl group, or a benzyl group, which may be identical or different. R^{6′} is the same as the above.

In another example, R represents a methyl group. R¹ represents a hydrogen atom. R² represents a hydroxymethyl group, NHR³, or SO₂NR⁴R^{4′}. R³ represents a hydrogen atom, a methyl group, SO₂R⁵, a formyl group, or CONHR^{6′}. Any one of R⁴ and R^{4′} represents a hydrogen atom, and the other represents a hydrogen atom, a lower alkyl group or a benzyl group. R⁵ represents a lower alkyl group, a benzyl group, or a dimethylamino group. R^{6′} is the same as the above.

In another example, R represents a methyl group. R¹ represents a halogen atom, or a hydroxyl group. R² represents NHSO₂R⁵, or SO₂NR⁴R^{4′}. R⁵ represents a lower alkyl group, a benzyl group, or NR⁴R^{4′}. R⁴ and R^{4′} respectively represent a hydrogen atom, a lower alkyl group, or a benzyl group, which may be identical or different.

In another example, R represents a methyl group. R¹ represents a fluorine atom, a chlorine atom, or a hydroxyl group. R² represents NHSO₂R⁵, or SO₂NR⁴R^{4′}. Any one of R⁴ and R^{4′} represents a hydrogen atom, and the other represents a hydrogen atom, a lower alkyl group or a benzyl group. R⁵ represents a lower alkyl group, a benzyl group, or a dimethylamino group.

In another example, R represents a methyl group. R¹ represents a hydrogen atom, a halogen atom, or a hydroxyl group. R² represents a hydrogen atom.

In another example, R represents a methyl group. R¹ represents a hydrogen atom, a fluorine atom, a chlorine atom, or a hydroxyl group. R² represents a hydrogen atom.

In another example, R represents a methyl group. R¹ represents a hydrogen atom, a halogen atom, a hydroxyl group, an amino group, or a hydroxymethyl group. R² represents NHR³, or SO₂NR⁴R^{4′}. R³ represents SO₂R⁵. R⁵ represents a lower alkyl group, a benzyl group, or NR⁴R^{4′}. R⁴ and R^{4′} respectively represent a hydrogen atom, a lower alkyl group, or a benzyl group, which may be identical or different.

Examples of compounds which are represented by the above formula (IV) include

(R)-N-[5-[2-[2-(9H-carbazole-2-yloxy)ethylamino]-1-hydroxyethyl]-2-hydroxyphenyl]methansulfonamide;

(S)-N-[5-[2-[2-(9H-carbazole-2-yloxy)ethylamino]-1-hydroxyethyl]-2-hydroxyphenyl]methansulfonamide;

N-[5-[2-[2-(9H-carbazole-2-yloxy)ethylamino]-1-hydroxyethyl]-2-hydroxyphenyl]methansulfonamide;

N-[5-[2-[2-(3-hydroxy-9H-carbazole-2-yloxy)ethylamino]-1-hydroxyethyl]-2-hydroxyphenyl]methansulfonamide;

N-[5-[2-[2-(3-amino-9H-carbazole-2-yloxy)ethylamino]-1-hydroxyethyl]-2-hydroxyphenyl]methansulfonamide;

N-[5-[2-[2-(6-amino-9H-carbazole-2-yloxy)ethylamino]-1-hydroxyethyl]-2-hydroxyphenyl]methansulfonamide;

N-[5-[2-[2-(6-hydroxy-9H-carbazole-2-yloxy)ethylamino]-1-hydroxyethyl]-2-hydroxyphenyl]methansulfonamide;

(R)-N-[3-[2-[2-(9H-carbazole-2-yloxy)ethylamino]-1-hydroxyethyl]phenyl]methansulfonamide;

(S)-N-[3-[2-[2-(9H-carbazole-2-yloxy)ethylamino]-1-hydroxyethyl]phenyl]methansulfonamide;

N-[3-[2-[2-(9H-carbazole-2-yloxy)ethylamino]-1-hydroxyethyl]phenyl]methansulfonamide;

N-methyl-3-[2-[2-(9H-carbazole-2-yloxy)ethylamino]-1-hydroxyethyl]benzenesulfonamide;

N-methyl-[5-[2-[2-(9H-carbazole-2-yloxy)ethylamino]-1-hydroxyethyl]-2-hydroxy]benzenesulfonamide;

(R)-N-[5-[2-[2-(dibenzofuran-3-yloxy)ethylamino]-1-hydroxyethyl]-2-hydroxyphenyl]methansulfonamide;

(S)-N-[5-[2-[2-(dibenzofuran-3-yloxy)ethylamino]-1-hydroxyethyl]-2-hydroxyphenyl]methansulfonamide;

N-[5-[2-[2-(dibenzofuran-3-yloxy)ethylamino]-1-hydroxyethyl]-2-hydroxyphenyl]methansulfonamide;

N-[5-[2-[2-(9H-carbazole-2-yloxy)ethylamino]-1-hydroxyethyl]-2-(benzyloxy)phenyl]methansulfonamide;

N-[5-[2-[2-(9H-carbazole-2-yloxy)ethylamino]-1-hydroxyethyl]-2-chlorophenyl]methansulfonamide;

N-[5-[2-[2-(9H-carbazole-2-yloxy)ethylamino]-1-hydroxyethyl]-2-fluorophenyl]methansulfonamide;

N-[3-[2-[2-(dibenzofuran-3-yloxy)ethylamino]-1-hydroxyethyl]phenyl]methansulfonamide;

N-[5-[2-[2-(7-acetylaminofluorene-2-yloxy)ethylamino]-1-hydroxyethyl]-2-hydroxyphenyl]methansulfonamide;

N-[5-[2-[2-(7-aminofluorene-2-yloxy)ethylamino]-1-hydroxyethyl]-2-hydroxyphenyl]methansulfonamide;

N-[3-[2-[2-(7-acetylaminofluorene-2-yloxy)ethylamino]-1-hydroxyethyl]phenyl]methansulfonamide;

N-[3-[2-[2-(7-aminofluorene-2-yloxy)ethylamino]-1-hydroxyethyl]phenyl]methansulfonamide;

N-[5-[2-[2-(9H-carbazole-2-yloxy)ethylamino]-1-hydroxyethyl]-2-hydroxyphenyl]formamide;

N-[3-[2-[2-(9H-carbazole-2-yloxy)ethylamino]-1-hydroxyethyl]phenyl]formamide;

N-[3-[2-[[1-(9H-carbazole-2-yloxy)propane-2R-yl]amino]-1-hydroxyethyl]phenyl]methansulfonamide;

2-[N-[2-(9H-carbazole-2-yloxy)ethyl]amino]-1-(4-hydroxy-3-nitrophenyl)ethanol;

2-[N-[2-(9H-carbazole-2-yloxy)ethyl]amino]-1-(3-amino-4-hydroxyphenyl)ethanol;

N-[5-[2-[2-(9H-carbazole-2-yloxy)ethylamino]-1-hydroxyethyl]-2-(benzyloxy)phenyl]urea;

N-[5-[2-[2-(9H-carbazole-2-yloxy)ethylamino]-1-hydroxyethyl]-2-hydroxyphenyl]urea;

N-[5-[2-[2-(9H-carbazole-2-yloxy)ethylamino]-1-hydroxyethyl]-2-(benzyloxy)phenyl]formamide;

N′-[5-[2-[2-(9H-carbazole-2-yloxy)ethylamino]-1-hydroxyethyl]-2-(benzyloxy)phenyl]-N,N-dimethylsulfamide;

N′-[5-[2-[2-(9H-carbazole-2-yloxy)ethylamino]-1-hydroxyethyl]-2-hydroxyphenyl]-N,N-dimethylsulfamide;

2-[N-[2-(9H-carbazole-2-yloxy)ethyl]amino]-1-(3-(methylamino)-4-(benzyloxy)phenyl)ethanol;

2-[N-[2-(9H-carbazole-2-yloxy)ethyl]amino]-1-(3-(methylamino)-4-hydroxyphenyl)ethanol;

N-[5-[2-[2-(9H-carbazole-2-yloxy)ethylamino]-1-hydroxyethyl]-2-hydroxyphenyl]-2-propanesulfonamide;

2-[N-[2-(9H-carbazole-2-yloxy)ethyl]amino]-1-(3-nitrophenyl)ethanol;

N′-[3-[2-[2-(9H-carbazole-2-yloxy)ethylamino]-1-hydroxyethyl]phenyl]-N,N-dimethylsulfamide;

2-[N-[2-(9H-carbazole-2-yloxy)ethyl]amino]-1-(3-aminophenyl)ethanol;

2-[N-[2-(9H-carbazole-2-yloxy)ethyl]amino]-1-[3-(hydroxymethyl)-4-hydroxyphenyl]ethanol;

N-[5-[2-[2-(9H-carbazole-2-yloxy)ethylamino]-1-hydroxyethyl]-3-hydroxyphenyl]methansulfonamide;

N-[3-[2-[2-(9H-carbazole-2-yloxy)ethylamino]-1-hydroxyethyl]-2-hydroxyphenyl]methansulfonamide;

N-[3-[2-[2-(9H-carbazole-2-yloxy)ethylamino]-1-hydroxyethyl]-4-hydroxyphenyl]methansulfonamide;

(R)-N40 -[5-[2-[2-(9H-carbazole-2-yloxy)ethylamino]-1-hydroxyethyl]-2-hydroxyphenyl]-N,N-dimethylsulfamide;

(S)-N′-[5-[2-[2-(9H-carbazole-2-yloxy)ethylamino]-1-hydroxyethyl]-2-hydroxyphenyl]-N,N-dimethylsulfamide;

N-[3-[2-[2-(6-acetylamino-9H-carbazole-2-yloxy)ethylamino]-1-hydroxyethyl]phenyl]methansulfonamide;

N-[5-[2-[2-(6-acetylamino-9H-carbazole-2-yloxy)ethylamino]-1-hydroxyethyl]-2-hydroxyphenyl]methansulfonamide;

(R)-N-[5-[2-[2-(9H-carbazole-2-yloxy)ethylamino]-1-hydroxyethyl]-2-fluorophenyl]methansulfonamide;

(S)-N-[5-[2-[2-(9H-carbazole-2-yloxy)ethylamino]-1-hydroxyethyl]-2-fluorophenyl]methansulfonamide;

(R)-N-[5-[2-[2-(9H-carbazole-2-yloxy)ethylamino]-1-hydroxyethyl]-2-chlorophenyl]methansulfonamide;

(S)-N-[5-[2-[2-(9H-carbazole-2-yloxy)ethylamino]-1-hydroxyethyl]-2-chlorophenyl]methansulfonamide;

N,N-dimethyl-[5-[2-[2-(9H-carbazole-2-yloxy)ethylamino]-1-hydroxyethyl]-2-hydroxy]benzensulfonamide;

N-[5-[2-[2-(9H-carbazole-2-yloxy)ethylamino]-1-hydroxyethyl]-2-iodophenyl]methansulfonamide;

N′-[5-[2-[2-(9H-carbazole-2-yloxy)ethylamino]-1-hydroxyethyl]-2-fluorophenyl]-N,N-dimethylsulfamide;

N′-[5-[2-[2-(9H-carbazole-2-yloxy)ethylamino]-1-hydroxyethyl]-2-chlorophenyl]-N,N-dimethylsulfamide;

(R)-N-[5-[2-[2-(9H-carbazole-2-yloxy)ethylamino]-1-hydroxyethyl]-2-hydroxy]benzenesulfonamide;

(R)-N-[5-[2-[2-(9H-carbazole-2-yloxy)ethylamino]-1-hydroxyethyl]-2-(hydroxymethyl)phenyl]methansulfonamide;

(R)-N-[3-[2-[2-(dibenzofuran-3-yloxy)ethylamino]-1-hydroxyethyl]phenyl]methansulfonamide;

N′-[5-[2-[2-(dibenzofuran-3-yloxy)ethylamino]-1-hydroxyethyl]-2-hydroxyphenyl]-N,N-dimethylsulfamide;

(R)-N′-[5-[2-[2-(dibenzofuran-3-yloxy)ethylamino]-1-hydroxyethyl]-2-hydroxyphenyl]-N,N-dimethylsulfamide;

(S)-N′-[5-[2-[2-(dibenzofuran-3-yloxy)ethylamino]-1-hydroxyethyl]-2-hydroxyphenyl]-N,N-dimethylsulfamide;

N-[5-[2-[2-(dibenzofuran-3-yloxy)ethylamino]-1-hydroxyethyl]-2-fluorophenyl]methansulfonamide;

N-[5-[2-[2-(dibenzofuran-3-yloxy)ethylamino]-1-hydroxyethyl]-2-chlorophenyl]methansulfonamide;

N-[5-[2-[2-(dibenzothiophene-3-yloxy)ethylamino]-1-hydroxyethyl]-2-hydroxyphenyl]methansulfonamide;

N′-[5-[2-[2-(dibenzothiophene-3-yloxy)ethylamino]-1-hydroxyethyl]-2-hydroxyphenyl]-N,N-dimethylsulfamide;

N-[3-[2-[2-(dibenzothiophene-3-yloxy)ethylamino]-1-hydroxyethyl]phenyl]methansulfonamide;

(R)-N-[5-[2-[2-(dibenzothiophene-3-yloxy)ethylamino]-1-hydroxyethyl]-2-hydroxyphenyl]methansulfonamide;

N-[5-[2-[2-(dibenzothiophene-3-yloxy)ethylamino]-1-hydroxyethyl]-2-fluorophenyl]methansulfonamide;

N-[5-[2-[2-(dibenzothiophene-3-yloxy)ethylamino]-1-hydroxyethyl]-2-chlorophenyl]methansulfonamide;

N-[5-[2-[2-(7-aminofluorene-2-yloxy)ethylamino]-1-hydroxyethyl]-2-hydroxyphenyl]methansulfonamide;

N′-[5-[2-[2-(7-acetylaminofluorene-2-yloxy)ethylamino]-1-hydroxyethyl]-2-hydroxyphenyl]-N,N-dimethylsulfamide;

N′-[5-[2-[2-(9H-carbazole-2-yloxy)ethylamino]-1-hydroxyethyl]-2-aminophenyl]-N-bezyl-N-methylsulfamide;

N-[5-[2-[2-(9H-carbazole-2-yloxy)ethylamino]-1-hydroxyethyl]-2-aminophenyl]methansulfonamide;

N-[5-[2-[2-(9H-carbazole-2-yloxy)ethylamino]-1-hydroxyethyl]-2-hydroxymethylphenyl]methansulfonamide;

N-[5-[2-[2-(9H-carbazole-2-yloxy)ethylamino]-1-hydroxyethyl]-2-bromophenyl]methansulfonamide;

N′-[5-[2-[2-(9H-carbazole-2-yloxy)ethylamino]-1-hydroxyethyl]-2-hydroxyphenyl]-N-bezyl-N-methylsulfamide;

N′-[5-[2-[2-(9H-carbazole-2-yloxy)ethylamino]-1-hydroxyethyl]-2-hydroxyphenyl]-N,N-diethylsulfamide;

N-[5-[2-[2-(9H-carbazole-2-yloxy)ethylamino]-1-methoxyethyl]-2-aminophenyl]methansulfonamide;

N-[5-[2-[2-(9H-carbazole-2-yloxy)ethylamino]-1-methoxyethyl]-2-hydroxyphenyl]methansulfonamide;

N-[5-[2-[2-(dibenzothiophene-3-yloxy)ethylamino]-1-methoxyethyl]-2-hydroxyphenyl]methansulfonamide;

2-[N-[2-(9H-carbazole-2-yloxy)ethyl]amino]-1-(4-hydroxyphenyl)ethanol;

2-[N-[2-(9H-carbazole-2-yloxy)ethyl]amino]-1-(2-fluorophenyl)ethanol;

2-[N-[2-(9H-carbazole-2-yloxy)ethyl]amino]-1-(2-hydroxyphenyl)ethanol;

(R,R)-2-[N-[1-(9H-carbazole-2-yloxy)propane-2-yl]amino]-1-phenyl]ethanol;

[2-[N-[2-(9H-carbazole-2-yloxy)ethyl]amino]-1-phenyl]ethanol;

(R)-[2-[N-[2-(9H-carbazole-2-yloxy)ethyl]amino]-1-phenyl]ethanol;

(S)-[2-[N-[2-(9H-carbazole-2-yloxy)ethyl]amino]-1-phenyl]ethanol;

2-[N-[2-(3-acetylamino-9H-carbazole-2-yloxy)ethyl]amino]-1-phenyl]ethanol;

2-[N-[2-(3-amino-9H-carbazole-2-yloxy)ethyl]amino]-1-phenyl]ethanol;

2-[N-[2-(3-hydroxy-9H-carbazole-2-yloxy)ethyl]amino]-1-phenyl]ethanol;

2-[N-[2-(6-amino-9H-carbazole-2-yloxy)ethyl]amino]-1-phenyl]ethanol;

2-[N-[2-(6-acetylamino-9H-carbazole-2-yloxy)ethyl]amino]-1-phenyl]ethanol;

[2-[N-[1-(9H-carbazole-2-yloxy)propane-2-yl]amino]-1-phenyl]ethanol;

[2-[N-[2-(dibenzofuran-3-yloxy)ethyl]amino]-1-phenyl]ethanol;

N-[5-[2-[2-(9H-carbazole-2-yloxy)ethylamino]-1-methoxyethyl]-2-hydroxy phenyl]methansulfonamide;

N-[5-[2-[2-(dibenzothiophene-3-yloxy)ethylamino]-1-methoxyethyl]-2-hydroxyphenyl]methansulfonamide;

N-[5-[2-[2-(9H-carbazole-2-yloxy)ethylamino]-1-methoxyethyl]-2-aminophenyl]methansulfonamide;

N-[5-[2-[2-(9H-carbazole-2-yloxy)ethylamino]-1-methoxyethyl]-2-chlorophenyl]methansulfonamide, etc.

The other examples of the β₃ adrenergic receptor agonist include the compounds which are represented by the following formulas (V), (VI), (VII) and (VIII). Note that the method of producing a compound which is represented by the following formula (IV), and the activity of the β₃ adrenergic receptor agonist are disclosed in WO98/03485, Drugs of the Future, vol. 16, p797-800 (1991), WO94/29290, and EP0659737, which are incorporated herein by reference.

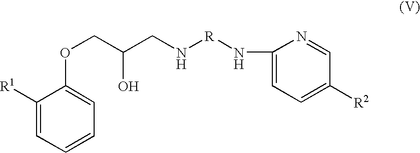

In the formula (V), R¹ represents a C₃₋₇ alkyl group or a C₃₋₇ cycloalkyl group; R² represents a hydrogen atom or an electron acceptor; and R represents a C₂₋₃ alkylene group.

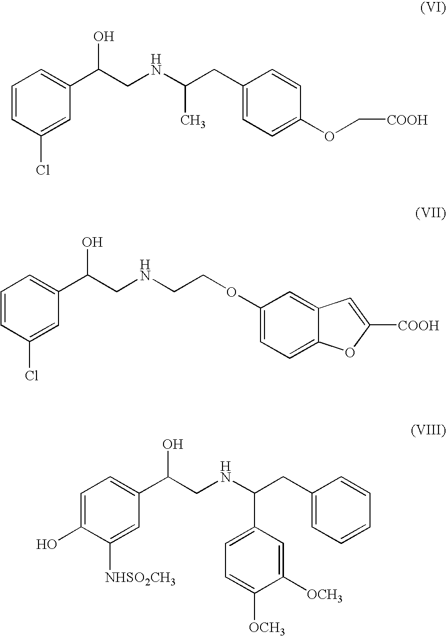

In cases where the compounds or the salts thereof represented by the following formulas (I) to (VIII) exist in the form of hydrate and/or solvate, the compounds or the salts thereof represented by the following formulas (I) to (VIII) may be hydrate and/or solvate.

In the present invention, a preferable compound for the β₃ adrenergic receptor agonist, which is administered to a patient with leptin resistance, has high selectivity to the β₃ adrenergic receptor. A preferable compound has a negligible action on the β₁ adrenergic receptor and β₂ adrenergic receptor in comparison with the action on the β₃ adrenergic receptor. Examples of the above compounds include AJ-9677, CL-316243, etc. By using a compound which has high selectivity to the β₃ adrenergic receptor, it becomes possible to suppress the side-effects, which may occur by the actions on the β₁ and β₃₂ adrenergic receptors, e.g., increased cardiac function, and carpopedal spasm.

Moreover, in the present invention, the β₃ adrenergic receptor agonist may be BRL-37344, BRL-35135, SB-418790, N-5984, YM178, a non-selective β receptor agonist, an α₁-adrenergic receptor blocker, carteolol, etc., and may be a combination of one or more than two β₃ adrenergic receptor agonists, which are described in the present specification.

Although, in the present invention, the β₃ adrenergic receptor agonist can be independently administered to a patient, it ordinarily is administered as a pharmaceutical composition, which is prepared by using one or more than two types of pharmaceutically acceptable carriers.

Examples of pharmaceutically acceptable carriers include water, pharmaceutically acceptable organic solvents, collagen, polyvinyl alcohol, polyvinyl pyrrolidone, carboxyvinyl polymers, sodium alginic acid, soluble dextran, sodium carboxymethyl starch, pectin, xanthan gum, gum arabic, casein, gelatin, agar, glycerin, propylene glycol, polyethylene glycol, vaseline, paraffin, stearyl alcohol, stearic acid, human serum albumin, mannitol, sorbitol, lactose, etc.

Examples of additives, which are used in formulation, include a filler, a bulking agent, a binder, a humidifying agent, a disintegrating agent, a surface active agent, a lubricant, a diluting agent, a stabilizer, a germicide, a buffering agent, an isotonic agent, a chelating agent, a pH control agent, a surfactant, etc. The above additives can be appropriately selected according to the form of a unit of administration, etc. for the preparation.

Examples of specific additives for stabilizers include: human serum albumin; L-amino acid such as glycine, cyctine, and glutaminic acid; monosaccharides such as glucose, mannose, galactose, and fluctose; sugar alcohol such as mannnitol, onositol, and xylitol; disaccharides such as sucrose, maltose, and lactose; polysaccharides such as dextran, hydroxypropyl starch, chondroitin sulfuric acid, hyaluronic acid, and the derivatives of said saccharides; and the cellulose derivatives such as methyl cellulose, ethyl cellulose, hydroxyethyl cellulose, hydroxypropyl cellulose, hydroxypropylmethyl cellulose, sodium carboxymethyl cellulose, etc.

Examples of surfactants include: polyoxyethylene glycol sorbitanalkylester, the polyoxyethylenealkylether type, sorbitan monoacylester type, and fatty acid glyceride type.

Examples of buffering agents include: boric acid, phosphoric acid, acetic acid, citric acid, ε-aminocaproic acid, glutamic acid, and the salts thereof, e.g., alkali metal salt or alkaline earth metal salt such as sodium salt, potassium salt, calcium salt, and magnesium salt.

Examples of isotonic agents include: sodium chloride, potassium chloride, saccharide, and glycerin.

Examples of chelating agents include: sodium edetic acid, and citric acid.

It is preferable to use the most effective administration path and formulation for the treatment. Examples of administration paths include non-oral paths such as nasal, intraencephalic, intraperitoneal, stomatic, respiratory-tract, intrarectal, subcutaneous, intramuscular and intravenal paths. Examples of formulations for administration include spray, capsules, tablets, granules, syrup, emulsions, suppositories, injections, ointments, tapes, etc.

Examples of formulations for oral administration include emulsion, syrup, capsules, tablets, powder, granule, etc. Liquid formulations such as syrup and emulsion can be produced by using the following additives: water; saccharides such as sucrose, sorbitol and fructose; the glycol type such as polyethylene glycol and propylene glycol; oil such as sesame oil, olive oil, and soybean oil; preservatives such as p-hydroxy benzoic acid ester types; and flavor additives such as strawberry flavor and peppermint. Capsules, tablets, powder, granules, etc. can be produced by using the following additives: diluting agents such as lactose, glucose, sucrose and mannitol; disintegrating agents such as starch and sodium alginic acid; lubricants such as magnesium stearic acid and talc; binders such as polyvinyl alcohol, hydroxypropyl cellulose and gelatine; surfactants such as fatty acid ester; and plasticizers such as glycerin.

Examples of appropriate formulations for non-oral administration include injections, suppositories, and spray. For example, an injection can be produced by using a carrier such as salt solution, glucose solution or the mixture thereof. A suppository can be produced by using a carrier such as cacao butter, hydrogenated fat, or carboxylic acid. A spray can be produced as an aerosol or dry powder, etc. by using lactose, glycerin, etc., which enable easy absorption of active ingredients without causing irritation of oral and respiratory-tract mucous membranes of a recipient by dispersing the active ingredients in the form of fine particles.

Although the dose and the number of administrations of β₃ adrenergic receptor agonists, which are effective in ameliorating leptin resistance of a patient, may vary depending on the targeted effect of treatment, the administration method, the period of treatment, age, body weight, etc., the dose can be ordinarily selected in an appropriate manner from 0.005-0.5 mg/kg per day, preferably from 0.01-0.1 mg/kg per day, and the number of administrations can be appropriately selected from 1-3 times per day, preferably from 1-2 times per day. If the dose is less than 0.005 mg/kg, the pharmaceutical composition becomes non-effective. However, if it is at 0.5 mg/kg, while there is a possibility of causing side effects, the pharmaceutical composition becomes effective. Moreover, if it is less than 0.01 mg/kg, the effect thereof becomes insignificant, whereas if it is more than 0.01 mg/kg, the effect thereof becomes effective. Furthermore, if the dose is more than 0.1 mg/kg, while being effective, the effect thereof is saturated with possibilities of causing side effects. Note that the above values are estimated based on the animal test, which will be described hereinafter, and in case of administration to a patient, the values after appropriate clinical trials and tests need to be determined according to the type of β₃ adrenergic receptor agonists.

In the present invention, it is preferable to administer leptin at the same time or after the administration of the β₃ adrenergic receptor agonist. As such, it becomes possible to ameliorate leptin resistance and enhance the action of leptin after the amelioration of leptin resistance.

Although it is possible to administer leptin independently, leptin is ordinarily administered as a pharmaceutical composition, which is prepared by using one or more than two types of pharmaceutically acceptable carriers. If leptin is administered at the same time with the β₃ adrenergic receptor agonist, a pharmaceutical composition, which comprises a β₃ adrenergic receptor agonist and leptin, can be used.

The dose and the number of administrations of leptin may vary depending on the targeted effect of treatment, the administration methods, the period of treatment, age, body weight, etc., the dose can be appropriately selected from 0.01-1.00 mg/kg per day, preferably from 0.01-0.30 mg/kg per day.

The amelioration of leptin resistance by the β₃ adrenergic receptor agonist is also useful for decreasing the weight-gaining effect of a pharmaceutical agent having a weight-gaining side-effect in addition to its intended prevention and/or treatment effects, e.g., diabetic drugs such as insulin, sulfonylurea agents, and drugs for enhancing the activity of PPARγ(Peroxisome Proliferator-activated Receptor γ) such as thiazolidine derivatives, alcohol, steroids, thyroid hormones, anti-histamine, and psychotropic drugs. It is preferable to administer an agent, which has a weight-gaining side effect, at the same time or after the administration of the β₃ adrenergic receptor agonist. If an agent, which has a weight-gaining side effect, is administered at the same time with the β₃ adrenergic receptor agonist, a pharmaceutical composition, which comprises a β₃ adrenergic receptor agonist and said agent having a weight-gaining side effect can be used.

### EXAMPLE

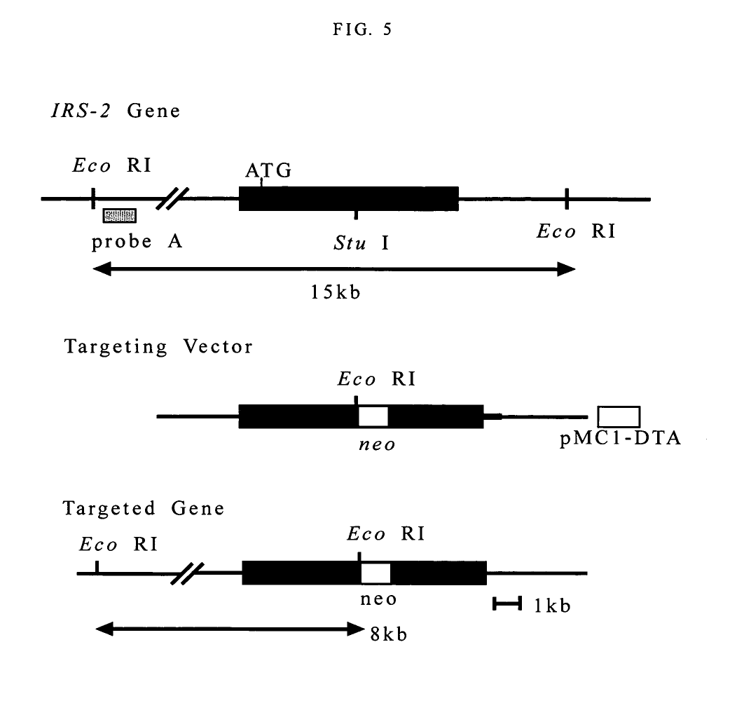

Insulin receptor substrate-2 (IR S-2) knockout mice were produced according to Kubota N. et al., Diabetes, November 2000, 49(11), 1880-9. Then, the IR S-2 gene (shown in the upper portion of FIG. 5) was screened from the mouse genome DNA library, and a targeting vector indicated in the middle of FIG. 5 was produced. The targeting vector was introduced into the TT2 ES cell, and ES cell clone with a recombinant was selected (shown in the lower portion of FIG. 5), which was introduced into the 8-celled embryo, thereby obtaining a chimera organism. A male chimeric organism was bred with a C57BL/6J female, thereby obtaining hetero-IR S-2 knockout mice, in which a recombinant gene has been introduced into the germ line. These hetero-knockout mice were bred with each other, thereby obtaining homo-IR S-2 knockout mice.

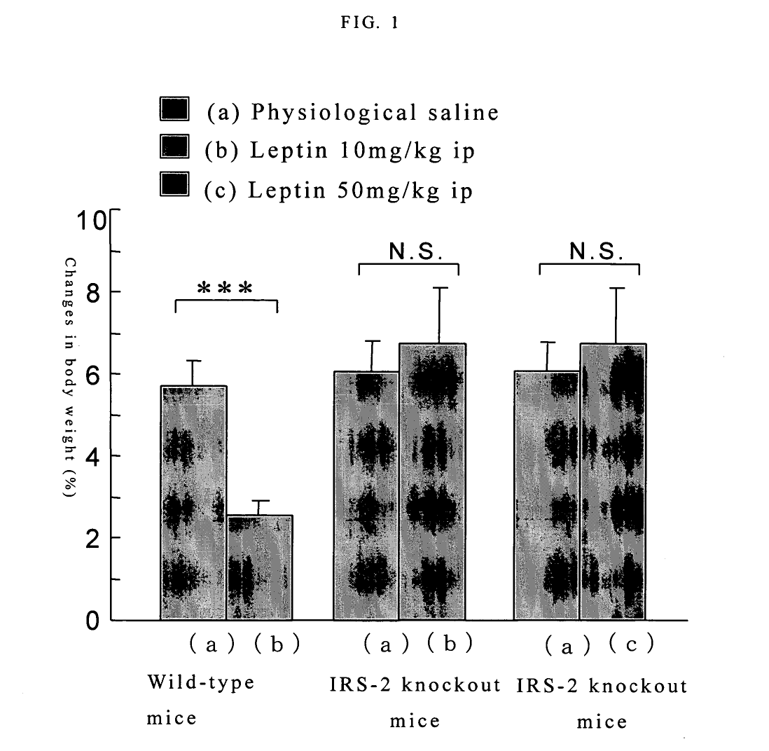

For the case where 10 mg/kg of body weight of leptin was intraperitoneally administered into the 8-week wild-type mouse, followed by food intake, the changes in body weight (%) after 12 hours of administration of leptin were measured. Moreover, (i) for the case where 10 mg/kg of body weight or 50 mg/kg of body weight of leptin was intraperitoneally administered into the 8-week IR S-2 knockout mouse, followed by food intake, the changes in body weight (%) after 12 hours of administration of leptin were measured. Furthermore, as a control, for the case where physiological saline was administered instead of leptin to the 8-week wild-type and IR S-2 knockout mice, the changes in body weight (%) were measured. The results are shown in FIG. 1. In FIG. 1, (a) is a result for the case in which the physiological saline was administered; (b) is a result for the case in which 10 mg/kg of body weight of leptin was intraperitoneally administered; and (c) is a result for the case in which 10 mg/kg of body weight of leptin was intraperitoneally administered. In FIG. 1, as well as in other figures, the values are expressed as means±S.E. (n=5−9); **, p<0.01; ***, p<0.001; and “N.S.” not significant. The statistical significances of the differences in body weight, etc. were determined by Student's t test (two-tailed).

As shown in FIG. 1, in the case of wild-type mice, a significant reduction in weight was observed by intraperitoneal administration of 10 mg/kg of body weight of leptin. However, in the case of IR S-2 knockout mice, a significant reduction in weight was not observed by intraperitoneal administration of 10 mg/kg of body weight or 50 mg/kg of body weight of leptin. Therefore, the results confirmed the fact that IR S-2 knockout mice show leptin resistance for leptin so peripherally administered.

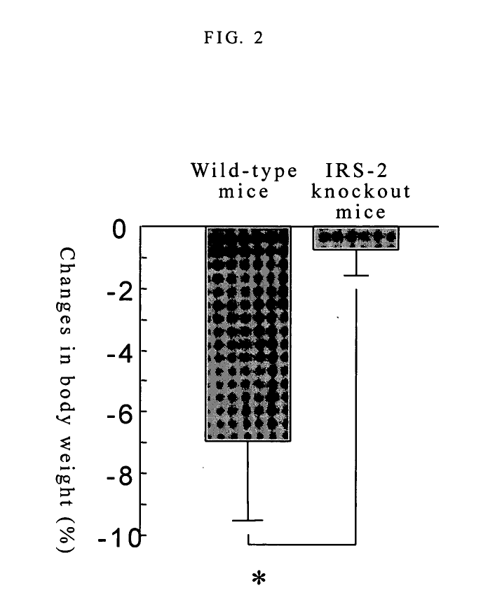

In a case where a canula was implanted ventricuarly to a 14-week wild-type mouse, and after starting the continuous ventricular administration of leptin (10 ng/hr), followed by food intake at will, the changes in body weigh (%) were measured after one week of said leptin administration. The results are shown in FIG. 2.

As shown in FIG. 2, in the case of wild-type mice, a significant reduction in weight was observed. However, in the case of IR S-2 knockout mice, a significant reduction in weight was not observed. Therefore, the results confirmed that IR S-2 knockout mice show leptin-resistance in the hypothalamus.

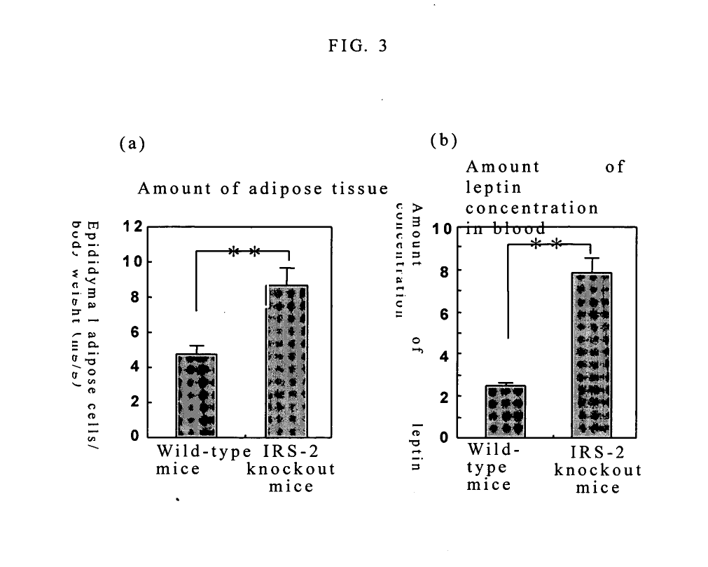

FIG. 3(a) and (b) shows the results of measurements of the amounts of adipose tissues (epididymal adipose cells/body weight (mg/g)) and the leptin concentrations in blood (ng/ml) with respect to the 8-week wild-type and IR S-2 knockout mice. The results shown in FIG. 3(a) and (b) confirmed that IR S-2 knockout mice were obese and that the level of leptin concentration in blood thereof was higher than that of wild-type mice.

For this case study, after the repeated oral administrations of 0.1 mg/kg of body weight of the compound expressed by the following formula (A) (referred to as “AJ-9677”) for 2 weeks, or after the repeated intraperitoneal administrations of the compound expressed by the following formula (B) (referred to as “CL-316243”) for 2 weeks, 20 mg/kg of body weight of leptin was intraperitoneally administered into the mouse, followed by food intake, and the changes in body weight (%) after 12 hours after the administration of leptin were measured.

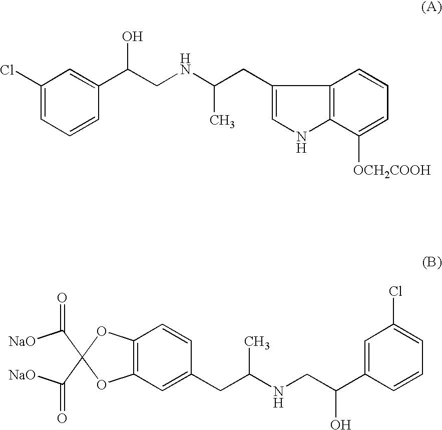

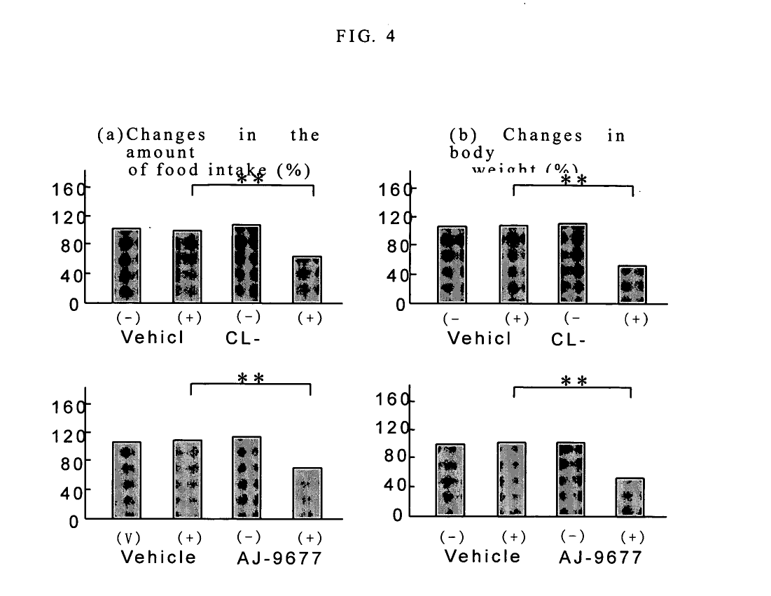

Moreover, as a control, for the case without intraperitoneal administration of leptin, in which 10 ml/kg of body weight of Tragacanth gum was repeatedly administered orally for 2 weeks, or in which 10 ml/kg of body weight of physiological saline solution was repeatedly intraperitoneally administered for 2 weeks instead of CL-316243, the changes in the amount of food intake, and the changes in body weight (%) were measured. The results are shown in FIG. 4. In FIG. 4, (a) indicates the changes in the amount of food intake (%); (b) indicates the changes in body weight (%); “CL-316243” shows the results for CL-316243 administration; and “AJ-9677” shows the results of AJ-9677 administration; “Vehicle” shows the results of Tragacanth gum or physiological saline administration; “+” shows the results of leptin administrations; and “−” shows the results of non-administration of leptin.

The results in FIG. 4 confirm the fact that, by administering AJ-9677 or CL-316243 (i.e., β₃ adrenergic receptor agonist), the leptin resistance in IR S-2 knockout mice is ameliorated, and the effect of peripheral administration of leptin is allowed to take place.

What is claimed is:

1. A method of ameliorating leptin resistance in a patient, which comprises a process of administering an effective dose of a β₃ adrenergic receptor agonist to said patient with leptin resistance.

2. The method of ameliorating leptin resistance according to claim 1, which further comprises a process of administering leptin to said patient.

3. The method of ameliorating leptin resistance according to claim 1, which further comprises a process of administering to said patient a pharmaceutical agent having a weight-gaining side effect in addition to its intended prevention or treatment effect.

4. The method of ameliorating leptin resistance according to claim 1, 2 or 3, wherein said β₃ adrenergic receptor agonist is a compound or the salt thereof, which is represented by the following formula (I): 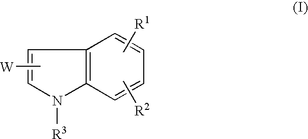
wherein, in the formula (I), R¹ represents the following groups, in which a hydroxyl group may be substituted respectively: a lower alkyl group, a phenylsulfonylamino group, a lower alkylsulfonylamino group, a mono- or di-lower alkylaminosulfonyl group, or a group which is selected from the following formulas (a) to (d); or R¹ and R² represents respectively a methylenedioxy group, which may be substituted by a carboxyl group or a lower alkoxycarbonyl group:
(a) a group which is represented by “—X—R^{a},” wherein X represents O, S, or NH, and R^{a} represents a hydrogen atom or a lower alkyl group, but if X═S, R^{a} is a lower alkyl group;
(b) a group which is represented by the following formula: 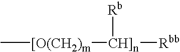
in which, R^{b} represents a hydrogen atom, a lower alkyl group, a lower alkoxycarbonyl group, or a carboxyl group; R^{bb} represents a lower alkoxycarbonyl group or a carboxyl group; m represents an integral number of 0 to 3; and n represents an integral number of 0 or 1;
(c) a group which is represented by “—O(CH₂)ₚ—R^{c},” wherein, R^{c} represents a lower alkanoyl group, a hydroxyl group, a cyano group, a phenyl group, a mono- or di-lower alkylaminocarbonyl group, or a group which is represented by the following formula: 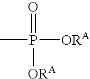
in which, p represents an integral number of 1 to 4; and R^{A} is a hydrogen atom or a lower alkyl group; and
(d) a group represented by “—Y—(CH₂)_{q}—R^{d},” wherein, Y represents NH or S; R^{d} represents a carboxyl group or a lower alkoxycarbonyl group; and q represents an integral number of 1 to 4;
wherein, R² represents a lower alkyl group, a hydroxyl group, or a lower alkoxy group, in which a hydrogen atom, a halogen atom, or a hydroxyl group may be substituted respectively; the same group as above (b) or (c); or R² and R¹ respectively represents a methylenedioxy group which may be substituted with a carboxyl group or a lower alkoxycarbonyl group;
wherein, R³ represents a hydrogen atom or a lower alkyl group; and
wherein, W represents a group of the following formula (I′), which is bonded to the second or the third position of the indole ring of the above formula (I): 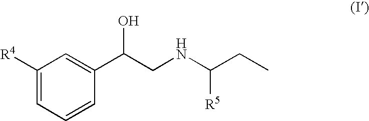
in which, R⁴ is a halogen atom or a trifluoromethyl group, and R⁵ is a lower alkyl group.

5. The method of ameliorating leptin resistance according to claim 1, 2 or 3, wherein said β₃ adrenergic receptor agonist is a compound or the salt thereof, which is represented by the following formula (II). 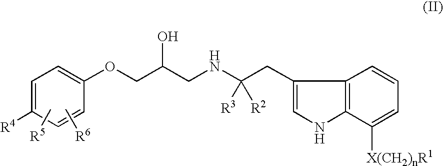
wherein, in the formula (II), X represents O, NH, or S;
wherein, R¹ represents a hydoroxy group; a lower alkoxycarbonyl group; a mono- or di-lower alkylaminocarbonyl group; a carbamoyl group; a cyclic aminocarbonyl group; a carboxyl group; a sulfamoyl group; a mono- or di-lower alkylaminosulfonyl group; a cyclic aminosulfonyl group; a sulfonic acid group; a sulfonic acid ester group; a phosphoric acid group; a phosphoric acid ester group; or a phenyl group unsubstituted or substituted by one or two types of substituents, which is selected from a halogen atom, a trifluoromethyl group, a lower alkyl group, a lower alkoxy group, a lower alkoxycarbonyl group, a carboxyl group, a nitro group, a cyano group, an amino group, and a mono- or di-lower alkylamino group;
wherein, R² and R³ represent respectively a hydrogen atom or a lower alkyl group, which may be identical or different;
wherein R⁴ represents a hydrogen atom or OR⁷, in which R⁷ represents a hydrogen atom; a lower alkyl group; a lower alkanoyl group; or a benzyl group unsubstituted or substituted by one or two types of substituents, which is selected from a halogen atom, a trifluoromethyl group, a lower alkyl group, a lower alkoxy group, a lower alkoxycarbonyl group, a carboxyl group, a nitro group, a cyano group, an amino group, and a mono- or di-lower alkylamino group;
wherein R⁵ represents a hydrogen atom, a halogen atom, a lower alkyl group, a lower alkoxy group, a hydroxy group, a nitro group, a cyano group, an amino group, a mono- or di-lower alkylamino group, a lower alkylsulfonylmino group, or a lower alkylsulfonyl group; and
wherein R⁶ represents a hydrogen atom, a halogen atom, a lower alkyl group, a lower alkoxy group, a hydroxy group, a nitro group, a cyano group, an amino group, a formylamino group, a mono- or di-lower alkylamino group, a lower alkylsulfonylamino group, or a lower alkylsulfonyl group; and
wherein n represents 1, 2, 3, 4 or 5.

6. The method of ameliorating leptin resistance according to claim 1, 2 or 3, wherein said β₃ adrenergic receptor agonist is a compound or the salt thereof, which is represented by the following formula (III). 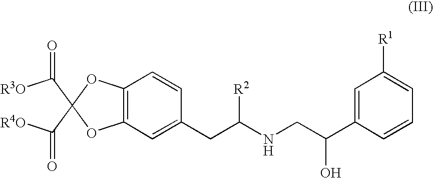
wherein, in the formula (III), R¹ represents a halogen atom; R² represents a hydrogen atom or a lower alkyl group; R³ and R⁴ represent respectively a hydrogen atom or a lower alkyl group, which may be identical or different.

7. The method of ameliorating leptin resistance according to claim 1, 2 or 3, wherein said β₃ adrenergic receptor agonist is a compound or the salt thereof, which is represented by the following formula (IV): 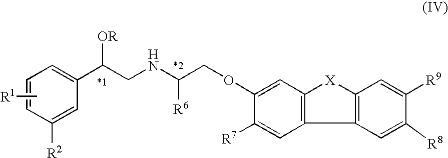
wherein, in the above formula (IV), R represents a hydrogen atom or a methyl group; R¹ represents a hydrogen atom, a halogen atom, a hydroxyl group, a benzyloxy group, an amino group, or a hydroxymethyl group; R² represents a hydrogen atom, a hydroxymethyl group, NHR³, SO₂NR⁴R^{4′}, or a nitro group; R³ represents a hydrogen atom, a methyl group, SO₂R⁵, a formyl group, or CONHR^{6′}; R⁵ represents a lower alkyl group, a benzyl group, or NR⁴R^{4′}; R⁴ and R^{4′} respectively represents a hydrogen atom, a lower alkyl group, or a benzyl group, which may be identical or different; R^{6′} represents a hydrogen atom or a lower alkyl group; R⁶ represents a hydrogen atom or a lower alkyl group; X represents a secondary nitrogen atom, an oxygen atom, a sulfur atom, or a methylene group; in cases where X is a secondary nitrogen atom, an oxygen atom, or a sulfur atom, R⁹is a hydrogen atom, and one of R⁷ and R⁸ is a hydrogen atom and the other is a hydrogen atom, an amino group, an acetylamino group or a hydroxyl group; and in cases where X is a methylene group, both R⁷ and R⁸ are hydrogen atoms, and R⁹ is a hydrogen atom, an amino group, an acetylamino group or a hydroxyl group; *1 represents an asymmetric carbon atom; and in cases where R⁶ is a lower alkyl group, *2 represents an asymmetric carbon atom.

8. A pharmaceutical composition comprising a β₃ adrenergic receptor agonist in a therapeutically effective amount capable of ameliorating leptin resistance.

9. A pharmaceutical composition according to claim 8, which further comprises leptin.

10. A pharmaceutical composition according to claim 8, which further comprises a pharmaceutical agent having a weight-gaining side effect in addition to its intended prevention or treatment effect.
